(19) United States
(12) Patent Application Publication
(10) Pub. No.: US 2026/0295230 A1
(43) Pub. Date: Oct. 1, 2026

(54) Tattooing Apparatus Having Linear Drive Mechanism with Magnetic Stabilisation and Dynamic Acceleration Recess

(71) Applicant: OLEG MOZHEY, Wroclaw (PL)

(72) Inventor: OLEG MOZHEY, Wroclaw (PL)

(21) Appl. No.: 19/561,636

(22) Filed: Mar. 10, 2026

Related U.S. Application Data
(60) Provisional application No. 63/777,401, filed on Mar. 25, 2025.

(51) Int. Cl. A61M 37/00

(52) U.S. Cl. CPC A61M 37/0076

(57) ABSTRACT

A tattooing apparatus has a drive mechanism that converts motor rotation into linear needle reciprocation through a sliding block mounted on a pair of parallel guide rods for constrained linear travel. An eccentric body mounted on the motor output shaft carries a bearing on a radially offset bearing seat, and the outer ring of the bearing engages the sliding block to drive it in reciprocation along the guide rods. Permanent magnets disposed in the sliding block and in the housing are arranged in a like-pole repulsive orientation to generate a magnetic preload that stabilises the sliding block during operation. A recess formed in an upper surface of the sliding block receives a portion of the bearing at the top-dead-centre position of the eccentric body, the recess having a profiled internal surface that produces an increased downward acceleration of the sliding block during the initial portion of the downstroke.

### CROSS-REFERENCE TO RELATED APPLICATIONS

This application claims the benefit of U.S. Provisional Application No. 63/777,401, filed on or about Mar. 25, 2025, the entire disclosure of which is hereby incorporated by reference.

### FIELD OF INVENTION

The present invention relates to drive mechanisms for tattoo devices, and more particularly to a linear reciprocating drive mechanism employing a slider-based transmission with magnetic stabilisation and a recess geometry that modifies the acceleration profile of the needle stroke.

### BACKGROUND

Tattoo machines operate by driving a needle or group of needles in rapid linear reciprocation to implant pigment into the dermis. The quality of the resulting tattoo depends in substantial part on the consistency, speed, and force profile of the needle stroke. A stroke that is too slow or too variable in force produces uneven pigment deposition, requiring the operator to make repeated passes over the same area and increasing trauma to the skin. A stroke that delivers excessive force causes unnecessary tissue damage and prolongs healing. The drive mechanism of the tattoo machine is therefore a critical determinant of both the quality of the tattoo and the comfort of the client.

Rotary tattoo machines, which have largely superseded traditional electromagnetic coil machines in professional use, convert the continuous rotation of a DC motor into linear reciprocation of a needle bar or needle cartridge. The conversion is typically accomplished through a crank, cam, or eccentric mechanism that translates the circular motion of the motor output shaft into a back-and-forth linear displacement. While the general principle is well established, the specific mechanical arrangement by which the rotary-to-linear conversion is achieved varies considerably among existing machines, and each arrangement presents its own set of trade-offs in respect of stroke consistency, vibration, friction, wear, and compactness.

Crank-and-linkage mechanisms are among the most common rotary-to-linear conversion arrangements in existing tattoo machines. In a typical configuration, a crank arm extends radially from the motor shaft and is connected through a linkage or connecting rod to a needle bar or piston that reciprocates within the housing. Slotted-link variants employ a slot formed in a reciprocating member through which a crank pin or bearing passes, the pin travelling along the slot as the crank rotates and thereby driving the reciprocating member in linear motion. These mechanisms are effective but introduce nonlinearities in the motion profile, generate vibration due to the mass of the rotating and reciprocating linkage components, and are subject to friction and wear at the sliding interfaces of the linkage joints. Mass-balancing techniques have been employed to reduce the vibrational effects of the rotating components, but these add complexity and bulk to the mechanism without addressing the underlying friction and wear characteristics of the sliding contact surfaces.

Slider-based drive mechanisms offer an alternative to crank-and-linkage arrangements. In a slider-based system, a sliding block is constrained to move along a linear guide and is driven in reciprocation by an eccentric element that contacts the slider directly or through an intermediate bearing. The linear guide may take the form of a groove or channel machined into the housing, or a pair of rods on which the slider rides. Slider-based mechanisms can provide more consistent linear motion than crank-and-linkage systems because the sliding block is constrained against rotational displacement and follows a true linear path defined by the guide geometry. However, existing slider-based arrangements in tattoo machines have been limited by the need for lubrication at the slider-to-guide interface, lateral instability of the slider during high-speed reciprocation, and susceptibility to wear-induced dimensional changes that degrade stroke precision over the service life of the machine. Furthermore, the acceleration profile of the needle stroke in existing slider-based mechanisms is determined entirely by the geometry of the eccentric and the clearance relationships between the eccentric element and the slider, offering limited scope for tuning the force characteristics of the stroke to match the requirements of different tattooing techniques.

It is known in the art to employ permanent magnets in tattoo machines for dampening or modifying the oscillatory motion of a reciprocating needle assembly. U.S. Pat. No. 9,393,395 to Chen et al. discloses a tattoo machine in which a pair of magnets is disposed axially within the main housing at one end of a cartridge assembly to dampen oscillating movement of the cartridge and needle bar. The first magnet moves synchronously with the cartridge and the second magnet is mounted to the housing, with the axial distance between the magnets being adjustable to tune the dampening force. The cartridge itself includes a primary piston having a slot that complementarily receives an eccentric member fixed to the motor output shaft, such that rotation of the eccentric member directly oscillates the cartridge. While this arrangement demonstrates the use of magnetic interaction to influence the behaviour of a reciprocating element in a tattoo machine, the magnetic dampening acts axially on the cartridge-and-needle-bar unit and the drive topology employs direct eccentric-to-slot engagement rather than a separately guided sliding block.

It is within this context that the present invention is provided.

### SUMMARY

According to one aspect of the present invention, there is provided a tattooing apparatus comprising a housing, a motor disposed within the housing and having an output shaft, and a pair of guide rods fixed within the housing and arranged parallel to one another. A sliding block is mounted on the guide rods for linear reciprocation therealong, the sliding block having rod-receiving bores through which the guide rods pass. An eccentric body is mounted on the output shaft of the motor for co-rotation therewith, the eccentric body having a bearing seat that is radially offset from an axis of the output shaft. A bearing is mounted on the bearing seat of the eccentric body, the bearing having an outer ring that engages the sliding block such that rotation of the output shaft causes the bearing to orbit about the axis of the output shaft and thereby drive the sliding block in linear reciprocation along the guide rods. A first permanent magnet is disposed in the sliding block and a second permanent magnet is disposed in the housing, the first and second permanent magnets being arranged in a like-pole repulsive orientation to generate a magnetic preload that stabilises the sliding block during reciprocation along the guide rods. A recess is formed in an upper surface of the sliding block and configured to receive a portion of the bearing at a top-dead-centre position of the eccentric body, the recess having a profiled internal surface such that, during rotation of the eccentric body past the top-dead-centre position, engagement of the bearing with the profiled internal surface produces an increased downward acceleration of the sliding block relative to a sliding block lacking the recess.

In one embodiment, the eccentric body has a central smooth bore configured to receive the output shaft, a lateral threaded hole oriented transversely to the central bore, and the bearing seat is formed as an offset cylindrical boss. A set screw is received in the lateral threaded hole and tightened against the output shaft to secure the eccentric body to the output shaft. In one embodiment, the sliding block is formed of a polymer composite material, and in a further embodiment the polymer composite material comprises a polyoxymethylene and polytetrafluoroethylene composite. In one embodiment, the guide rods are cylindrical and formed of hardened stainless steel having a polished finish. In one embodiment, the apparatus further comprises a battery cell disposed within the housing and electrically connected to the motor, such that the apparatus is operable as a wireless handheld device, and a battery module frame may be fixedly disposed within the housing to receive and locate the battery cell for user replacement without disassembly of the drive mechanism.

According to another aspect of the present invention, there is provided a linear drive mechanism for converting rotary motion into linear reciprocation, the mechanism comprising a pair of guide rods arranged parallel to one another, a sliding block mounted on the guide rods for linear reciprocation therealong, an eccentric body configured to be mounted on a rotary output shaft and having a bearing seat that is radially offset from a rotational axis of the output shaft, a bearing mounted on the bearing seat and having an outer ring that engages the sliding block to convert rotation of the output shaft into linear reciprocation of the sliding block along the guide rods, a magnetic preload system comprising a first permanent magnet associated with the sliding block and a second permanent magnet associated with a stationary structure adjacent to the sliding block arranged in a like-pole repulsive orientation to generate a stabilising force on the sliding block during reciprocation, and a recess formed in a surface of the sliding block that faces the bearing and configured to receive a portion of the bearing at a top-dead-centre position of the eccentric body, the recess having a profiled internal surface that produces an increased acceleration of the sliding block away from the bearing during rotation of the eccentric body past the top-dead-centre position.

According to a further aspect of the present invention, there is provided a method of operating a tattoo apparatus, the method comprising rotating an output shaft of a motor disposed within a housing to rotate an eccentric body mounted on the output shaft, the eccentric body carrying a bearing on a bearing seat that is radially offset from an axis of the output shaft, driving a sliding block in linear reciprocation along a pair of parallel guide rods fixed within the housing by engagement of an outer ring of the bearing with the sliding block as the bearing orbits about the axis of the output shaft, stabilising the sliding block during reciprocation by a repulsive magnetic interaction between a first permanent magnet disposed in the sliding block and a second permanent magnet disposed in the housing arranged in a like-pole orientation, and modifying an acceleration profile of the sliding block during a downstroke by receiving a portion of the bearing into a recess formed in an upper surface of the sliding block at a top-dead-centre position of the eccentric body, the recess having a profiled internal surface that produces an increased downward acceleration of the sliding block as the bearing transitions out of the recess during rotation past the top-dead-centre position.

### BRIEF DESCRIPTION OF THE DRAWINGS

Various embodiments of the invention are disclosed in the following detailed description and accompanying drawings.

FIG. 1 is an isometric cutaway view of the drive mechanism in accordance with one embodiment of the present invention, showing the eccentric body, bearing, guide rods, sliding block, and dynamic recess.

FIG. 2A is a longitudinal sectional view taken along the line A-A of the drive mechanism, showing the housing, guide rods, sliding block, eccentric body, bearing, and dynamic recess.

FIG. 2B is an enlarged detail view of the region identified by the detail circle C in FIG. 2A, showing the bearing partially received within the dynamic recess at a top-dead-centre position of the eccentric body.

FIG. 3 is a transverse sectional view taken along the line B-B of the drive mechanism, showing the housing, sliding block, and the magnet cavities of the magnetic stabilisation system.

FIG. 4A is a detail view of a portion of the housing showing the magnet cavity and retention fastener arrangement.

FIG. 4B is a detail view of the sliding block showing the corresponding magnet cavity and retention fastener arrangement.

FIG. 5 is an exploded view of the battery module arrangement, showing the housing, battery module frame, and replaceable battery cell.

FIG. 6 is an exploded isometric view of the complete tattoo apparatus, showing the assembly sequence and spatial relationships among all principal components.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

As used herein, the term “guide rods” refers to elongate members that define a linear path along which the sliding block reciprocates. The guide rods may be solid or hollow, and may have a circular, elliptical, or polygonal cross-section, though a circular cross-section is preferred. The term encompasses rods, shafts, rails, and pins regardless of the specific manufacturing process by which they are formed, provided that they serve the function of constraining the sliding block to linear travel.

As used herein, the term “sliding block” refers to the reciprocating component of the drive mechanism that is mounted on the guide rods for linear travel and that receives the driving force from the bearing. The sliding block may also be referred to in the art as a slider, piston, shuttle, or carriage. The sliding block is distinguished from a slotted link or connecting rod in that it is constrained to linear travel by the guide rods rather than being pivotally connected at one or both ends.

As used herein, the term “eccentric body” refers to a component that is mountable on a rotary shaft and that has a bearing seat whose axis is offset from the axis of the rotary shaft. The eccentric body may be formed as a single unitary piece or as an assembly of separately formed parts that are fixed together. The term encompasses components that may be referred to in the art as an eccentric, an eccentric cam, an eccentric disc, or an offset hub.

As used herein, the term “bearing seat” refers to a surface of the eccentric body on which the bearing is mounted. The bearing seat may be a cylindrical boss, a shoulder, a stepped diameter, or any other formation configured to locate and retain the bearing. The term “radially offset” in connection with the bearing seat means that the geometric centre of the bearing seat is spaced from the rotational axis of the output shaft by a nonzero distance, such that rotation of the eccentric body causes the bearing seat and the bearing mounted thereon to orbit about the rotational axis.

As used herein, the term “like-pole repulsive orientation” means an arrangement in which a north pole of one magnet faces a north pole of the other magnet, or a south pole of one magnet faces a south pole of the other magnet, such that the magnetic interaction between the two magnets is predominantly repulsive. The orientation need not be perfectly axially aligned; arrangements in which the like poles face one another within a deviation of up to approximately 30 degrees from direct opposition are within the scope of the term.

As used herein, the term “magnetic preload” refers to a force generated by the repulsive magnetic interaction between the first and second permanent magnets that acts on the sliding block in a direction having at least a lateral component relative to the direction of reciprocation. The magnetic preload biases the sliding block toward a equilibrium position on the guide rods and resists displacement of the sliding block from that equilibrium position. The term “stabilises” in connection with the magnetic preload means that the preload reduces vibration, resists lateral deviation, or maintains consistent stroke behaviour of the sliding block, or any combination thereof.

As used herein, the term “recess” refers to a concavity, pocket, depression, or shaped cavity formed in a surface of the sliding block. The recess is open toward the bearing such that a portion of the bearing may enter the recess during a portion of the eccentric cycle. The recess is distinguished from a through-hole or a slot in that it does not extend entirely through the sliding block.

As used herein, the term “profiled internal surface” refers to the surface that defines the interior geometry of the recess. The profiled internal surface is non-planar relative to the surrounding upper surface of the sliding block, such that the bearing undergoes a change in vertical displacement when transitioning through the recess that differs from the displacement that would occur on a flat surface. The profiled internal surface may be curved, angled, stepped, faceted, or any combination thereof. By way of non-limiting example, the profiled internal surface may be defined by a single radius of curvature, by a compound curve having two or more radii, by a spline-defined contour, by one or more angled flat faces meeting at an included angle, by a stepped profile, or by a compound profile combining curved and angled portions. The term does not require that the entire internal surface of the recess be curved or that the transition from the recess to the surrounding upper surface be smooth or tangent-continuous; abrupt transitions such as angular edges or steps are within the scope of the term.

As used herein, the term “top-dead-centre position” refers to the rotational position of the eccentric body at which the bearing seat is at its maximum displacement in the upward direction, corresponding to the point in the eccentric cycle at which the sliding block is at or near the uppermost extent of its reciprocating travel. The top-dead-centre position need not correspond to a mathematically exact maximum; the term encompasses a range of rotational positions in the immediate vicinity of the geometric maximum at which the bearing is in engagement with the recess.

As used herein, the term “increased downward acceleration” is a relative term meaning that the downward acceleration of the sliding block during rotation of the eccentric body past the top-dead-centre position is greater than the downward acceleration that would be produced by an otherwise identical mechanism in which the upper surface of the sliding block is flat and lacks the recess. The increase in acceleration results from the geometry of the profiled internal surface of the recess, which permits the bearing to undergo a greater change in vertical displacement per unit of rotational angle during the transition out of the recess than would occur on a flat surface.

As used herein, the term “transient floating condition” refers to a brief interval during the eccentric cycle in which the contact force between the outer ring of the bearing and the sliding block is reduced to substantially zero or to a value that is less than 20 percent of the peak contact force occurring during the downstroke. The transient floating condition occurs at or near the top-dead-centre position as the bearing enters the recess and the driving force on the sliding block is momentarily relieved.

As used herein, the term “self-lubricating” in reference to the sliding block or the polymer composite material means that the material of the sliding block has inherent lubricity sufficient to permit reciprocation of the sliding block on the guide rods without application of an external liquid or grease lubricant under normal operating conditions.

As used herein, the term “stationary structure” in the context of the magnetic preload system refers to any component of the mechanism that does not reciprocate with the sliding block. The stationary structure may be the housing, a sub-housing, a frame, a bracket, or any other component that is fixed relative to the guide rods during operation.

Unless expressly stated otherwise, words such as “a,” “an,” and “the” are intended to include both singular and plural forms, and the term “about” is intended to accommodate ±10% variations in stated values. Recitation of a range inherently includes all sub-ranges and individual values within that range. All exemplary materials, temperatures, and dimensions may be interchanged with other functionally equivalent counterparts unless contradicted by express language. The scope of the invention should therefore be construed in light of the appended claims, with these passages serving only to illustrate representative but non-limiting embodiments.

### Description of Drawings

The following detailed description is made with reference to the accompanying drawings, in which like reference numerals designate like parts throughout the several views. The described embodiments are illustrative and are not intended to limit the scope of the invention as defined by the appended claims. Dimensions, materials, and other specifics recited herein are given by way of example and not limitation, and one of ordinary skill in the art will recognise that modifications and substitutions may be made without departing from the spirit and scope of the invention.

The present invention is described herein in the context of a wireless handheld tattoo machine; however, the drive mechanism disclosed herein is not limited to tattooing applications and may be employed in any apparatus requiring the conversion of rotary motion into controlled linear reciprocation with low vibration, lubrication-free operation, and a tunable acceleration profile.

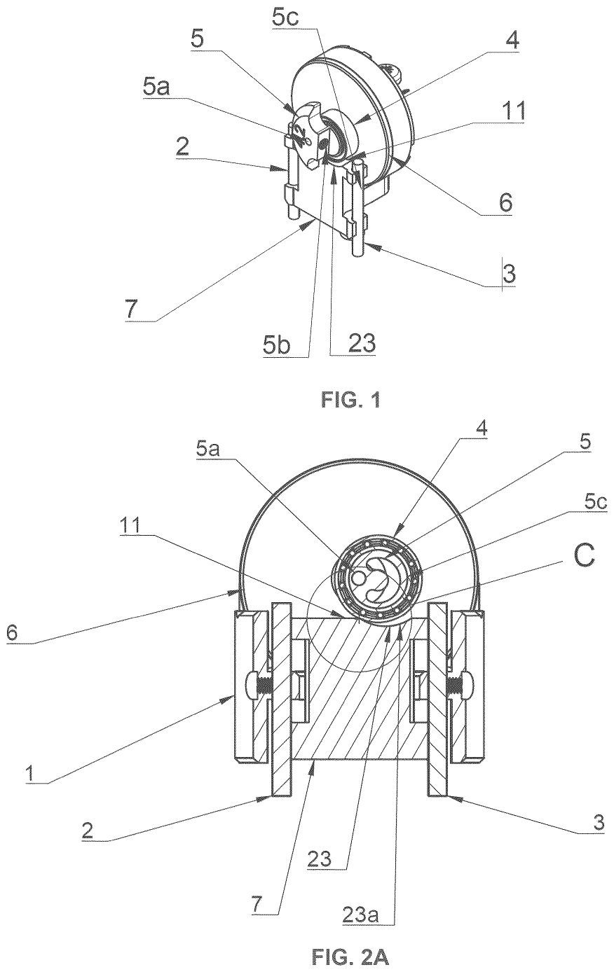

FIG. 1 is an isometric cutaway view of the drive mechanism in accordance with one embodiment of the present invention. The drive mechanism is configured to convert rotary motion from a motor 6 into linear reciprocation suitable for driving a tattoo needle cartridge. As shown, the motor 6 has an output shaft that extends forwardly through a motor front plate 4. The motor front plate 4 is a generally disc-shaped structural member that mounts within the housing and provides a stable mounting interface between the motor 6 and the remaining components of the drive mechanism.

An eccentric body 5 is mounted on the output shaft of the motor 6. The eccentric body 5 has a central smooth bore 5a sized to receive the motor output shaft, and a lateral threaded hole 5b oriented transversely to the axis of the central bore 5a. A set screw (not separately numbered) is received in the lateral threaded hole 5b and is tightened against the motor output shaft to fix the eccentric body 5 to the shaft for co-rotation therewith. The eccentric body 5 further includes an offset cylindrical bearing seat 5c that is spaced radially from the central bore 5a. The radial offset between the axis of the central bore 5a and the axis of the bearing seat 5c defines the eccentric radius, which in turn determines the stroke length of the reciprocating motion produced by the mechanism.

A bearing 11 is press-fit onto the offset bearing seat 5c such that the bearing 11 rotates with the eccentric body 5 about the axis of the motor output shaft. As the motor 6 rotates the output shaft, the eccentric body 5 co-rotates and the bearing 11 orbits about the shaft axis in a circular path whose radius corresponds to the eccentric radius defined by the offset of the bearing seat 5c.

The drive mechanism further includes a pair of parallel cylindrical guide rods 2, 3 that are fixed within the housing and extend generally parallel to the direction of intended linear reciprocation. A sliding block 7 is mounted on the guide rods 2, 3 for constrained linear travel therealong. The sliding block 7 has rod-receiving bores through which the guide rods 2, 3 pass, permitting the sliding block 7 to reciprocate along the guide rods 2, 3 while being restrained against lateral and rotational displacement.

The outer ring of the bearing 11 engages an upper interface region of the sliding block 7 such that orbital motion of the bearing 11 is converted into linear reciprocation of the sliding block 7 along the guide rods 2, 3. As the bearing 11 orbits, it pushes the sliding block 7 downwardly during the downstroke portion of the cycle and permits the sliding block 7 to return upwardly during the upstroke portion, producing continuous reciprocation.

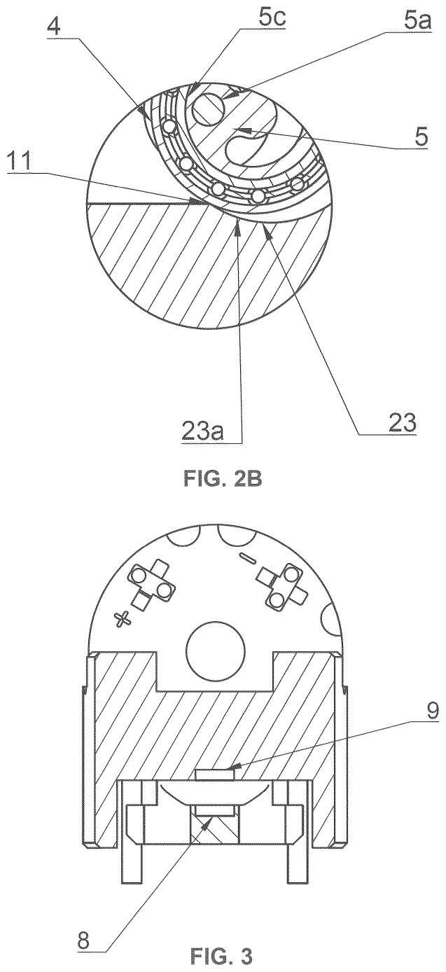

As further shown in FIG. 1, the sliding block 7 includes a dynamic recess 23 formed in an upper surface thereof. The dynamic recess 23 is a shaped cavity configured to receive a portion of the bearing 11 at or near the top-dead-centre position of the eccentric cycle. The geometry and function of the dynamic recess 23 are described in further detail below with reference to FIG. 2B.

FIG. 2A is a longitudinal sectional view taken along the line A-A of the drive mechanism, illustrating the structural relationships among the housing 1, the guide rods 2, 3, the sliding block 7, the eccentric body 5, the bearing 11, and the dynamic recess 23. The housing 1 is formed as a generally tubular enclosure having internal walls that define a cavity within which the drive mechanism components are received. As shown in section, the housing 1 includes guide rod mounting features on opposing internal walls thereof, through which the guide rods 2, 3 extend and are fixedly retained. The guide rods 2, 3 are oriented generally parallel to the longitudinal axis of the housing 1, and each guide rod is secured at its upper end to the housing 1 by a respective fastener visible in the sectional view.

The sliding block 7 is disposed between the internal walls of the housing 1 and is mounted on the guide rods 2, 3 for linear reciprocation therealong. In the sectional view of FIG. 2A, the cross-hatching of the sliding block 7 is distinguishable from that of the housing 1, indicating that the two components are formed of different materials. As described herein, the sliding block 7 is formed of a POM-PTFE polymer composite, whereas the housing 1 may be formed of a metal or rigid polymer.

The motor front plate 4 is visible in section above the sliding block 7 as a generally circular structural member through which the motor output shaft extends. The eccentric body 5 is mounted on the motor output shaft forwardly of the motor front plate 4. The central smooth bore 5a is visible in section as the bore through which the motor shaft passes, and the offset bearing seat 5c is shown as the eccentric boss portion that is spaced radially from the central bore 5a. The bearing 11 is seated on the offset bearing seat 5c, and the individual ball elements of the bearing 11 are visible within the bearing race in this sectional view.

A detail circle denoted C is indicated on FIG. 2A, identifying the region of interaction between the bearing 11 and the dynamic recess 23 that is shown at enlarged scale in FIG. 2B.

The dynamic recess 23 is formed in the upper surface of the sliding block 7 and is visible in the sectional view as a concavity opening upwardly toward the bearing 11. A profiled recess surface 23a defines the internal profile of the dynamic recess 23. The profiled recess surface 23a has a contour that is non-planar relative to the surrounding upper surface of the sliding block 7. In the embodiment shown, the profiled recess surface 23a has a smoothly arcuate profile, but the recess geometry is not limited to curved surfaces. The profiled recess surface 23a may alternatively be defined as an angled surface, a stepped surface, a faceted surface, a compound profile combining curved and angled portions, or any other non-flat geometry selected to achieve a desired acceleration characteristic during the downstroke.

FIG. 2B is an enlarged detail view corresponding to the region identified by the detail circle C in FIG. 2A, showing the interaction between the bearing 11 and the dynamic recess 23 at approximately the top-dead-centre position of the eccentric cycle. The enlarged view reveals the eccentric body 5 in section, with the central smooth bore 5a visible on one side and the offset bearing seat 5c on the other. The cross-hatching of the eccentric body 5 is shown in a different pattern from that of the sliding block 7 below, confirming that these are separate components formed of different materials.

The bearing 11 is shown seated on the offset bearing seat 5c, with the inner ring of the bearing 11 press-fit onto the bearing seat 5c, the ball elements disposed within the annular race, and the outer ring of the bearing 11 extending downwardly into the dynamic recess 23. At the top-dead-centre position depicted in FIG. 2B, the outer ring of the bearing 11 has entered the dynamic recess 23 such that a lower portion of the bearing 11 is received within the concavity defined by the profiled recess surface 23a.

During operation, as the eccentric body 5 rotates through the top-dead-centre position, the bearing 11 momentarily seats into the dynamic recess 23 and the contact force between the bearing 11 and the sliding block 7 is temporarily reduced. This momentary load reduction produces a transient condition in which the sliding block 7 is substantially unloaded by the bearing 11, creating a brief floating interval in which the upward driving force on the sliding block 7 approaches zero. As the eccentric body 5 continues to rotate past top-dead-centre, the bearing 11 traverses along the profiled recess surface 23a and re-engages the upper surface of the sliding block 7 outboard of the recess 23. The transition from the recessed position to the flat upper surface produces an accelerated onset of downward displacement of the sliding block 7 relative to a mechanism lacking such a recess. The net effect is a sharper initial downstroke acceleration, which enhances the velocity of the needle during the skin-penetration phase of the tattoo stroke, thereby improving the efficiency of pigment implantation while reducing the overall force duration and associated tissue trauma. This acceleration enhancement also permits the mechanism to operate effectively at lower motor voltages than would otherwise be required to achieve equivalent pigment deposition, reducing power consumption and extending battery life in wireless tattoo machine configurations.

FIG. 3 is a transverse sectional view taken along the line B-B of the drive mechanism, illustrating the spatial relationship between the housing 1, the sliding block 7, and the magnetic stabilisation system. The section is taken in a plane generally perpendicular to the longitudinal axis of the guide rods 2, 3, such that the housing 1 is shown in cross-section with its internal cavity visible. The motor front plate 4 is partially visible in the upper portion of the sectional view as a semi-circular section, with the motor shaft bore and various mounting features visible in the cut plane.

The housing 1 is shown in cross-hatched section on opposing sides of the sliding block 7, defining the internal walls within which the sliding block 7 reciprocates. The sliding block 7 is disposed centrally within the cavity of the housing 1, and the bores through which the guide rods 2, 3 pass are visible in the cut plane, confirming the parallel guide rod arrangement that constrains the sliding block 7 to linear travel.

As shown in FIG. 3, the housing 1 includes a magnet cavity 9 formed in an inner wall thereof. The magnet cavity 9 is a pocket or recess machined or moulded into the housing wall and oriented to face inwardly toward the sliding block 7. A permanent magnet is disposed within the magnet cavity 9 and is retained therein during operation. The sliding block 7 includes a corresponding magnet cavity 8 formed in a surface thereof that faces the inner wall of the housing 1 in which the magnet cavity 9 is formed. A permanent magnet is likewise disposed within the magnet cavity 8 of the sliding block 7.

The permanent magnets within the cavities 8 and 9 are arranged with like poles facing one another across the gap between the sliding block 7 and the inner wall of the housing 1, such that the magnets generate a repulsive force therebetween. This repulsive magnetic interaction produces a stabilising preload that biases the sliding block 7 away from the housing wall in which the magnet cavity 9 is located. The preload acts laterally on the sliding block 7 relative to the direction of reciprocation, thereby resisting lateral displacement of the sliding block 7 on the guide rods 2, 3 during high-speed operation. The magnetic preload reduces vibration, maintains consistent stroke behaviour across a range of operating speeds and needle cartridge loads, and ensures reliable startup of the reciprocating motion when the motor 6 is energised under cartridge load conditions that would otherwise tend to resist initial displacement of the sliding block 7.

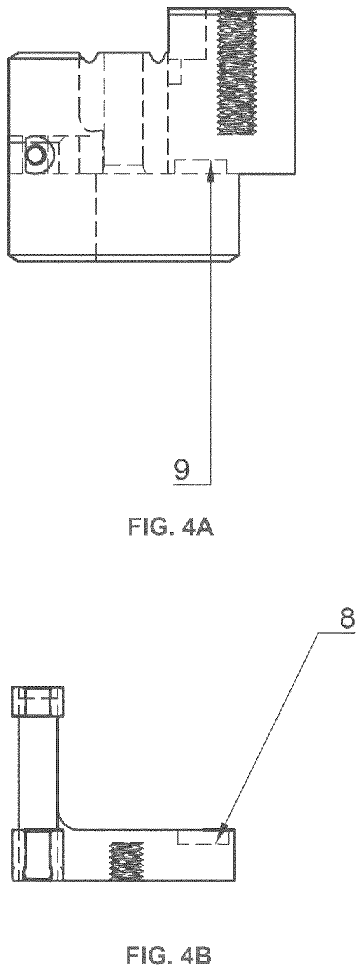

FIGS. 4A and 4B are detail views illustrating the magnet installation arrangement on the housing 1 and the sliding block 7, respectively.

FIG. 4A shows a portion of the housing 1 in isolation. The magnet cavity 9 is visible as a pocket formed in the inner wall of the housing 1. The internal profile of the magnet cavity 9 is indicated in dashed lines, showing the extent of the pocket within the housing wall. A retention fastener is received in a threaded bore that communicates with the magnet cavity 9, the retention fastener being tightened to secure the permanent magnet within the cavity 9 and to prevent dislodgment of the magnet during the vibrational loading associated with high-speed reciprocation of the drive mechanism. A guide rod bore is visible at one side of the housing portion, confirming the spatial relationship between the magnet cavity 9 and the guide rod mounting features of the housing 1.

FIG. 4B shows the sliding block 7 in isolation. The magnet cavity 8 is formed in a surface of the sliding block 7 that, when assembled, faces the inner wall of the housing 1 containing the magnet cavity 9. One of the guide rods 2 is shown passing through a rod-receiving bore of the sliding block 7, with a fastener visible at the upper end securing the guide rod within the housing. A retention fastener corresponding to that shown in FIG. 4A is received in a threaded bore communicating with the magnet cavity 8 of the sliding block 7, securing the permanent magnet within the cavity 8. The mirrored retention arrangements of FIGS. 4A and 4B ensure that both permanent magnets remain securely seated within their respective cavities 8, 9 throughout the full range of operating conditions, including sustained high-frequency reciprocation and the associated inertial and vibrational forces.

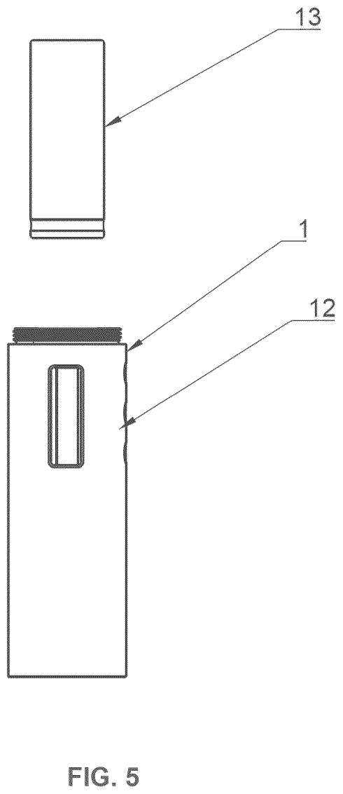

FIG. 5 is an exploded view of the battery module arrangement within the housing 1. The housing 1 is shown as the main tubular body of the tattoo machine, having a generally cylindrical exterior profile and an internal cavity configured to receive the drive mechanism components at one end and the battery module at the other. The housing 1 includes an open upper end through which the battery cell 13 is insertable, and a lower region within which the drive mechanism components described with reference to FIGS. 1 through 4B are received.

A battery module frame 12 is disposed within the housing 1. The battery module frame 12 is a structural insert having an open-frame construction that defines an internal receptacle sized to receive and locate the battery cell 13. The battery module frame 12 is fixedly mounted within the housing 1 and is not intended to be removed by the user during normal operation. A rectangular window is formed in one wall of the battery module frame 12, which may serve to permit electrical connection routing, provide visual access to the battery cell 13 for inspection purposes, or accommodate thermal dissipation during sustained operation of the motor 6. The battery module frame 12 provides structural support for the battery cell 13 and positions it in correct alignment with the electrical contacts of the motor 6 and any associated control circuitry disposed within the housing 1.

The battery cell 13 is a replaceable cylindrical electrochemical cell having a positive terminal at one end and a contact ring at the opposing end. The battery cell 13 is insertable into the battery module frame 12 from the upper end of the housing 1, and is removable by the user for replacement when the cell is depleted. A seal or retaining ring is visible at the junction between the battery cell 13 and the housing 1, which may serve to retain the battery cell 13 in position during use and to prevent ingress of debris into the internal cavity of the housing 1. The arrangement of a fixed battery module frame 12 with a user-replaceable battery cell 13 permits the operator to exchange a depleted cell for a fresh one in the field without requiring disassembly of the drive mechanism or any other internal component of the apparatus, thereby minimising downtime during extended tattoo sessions.

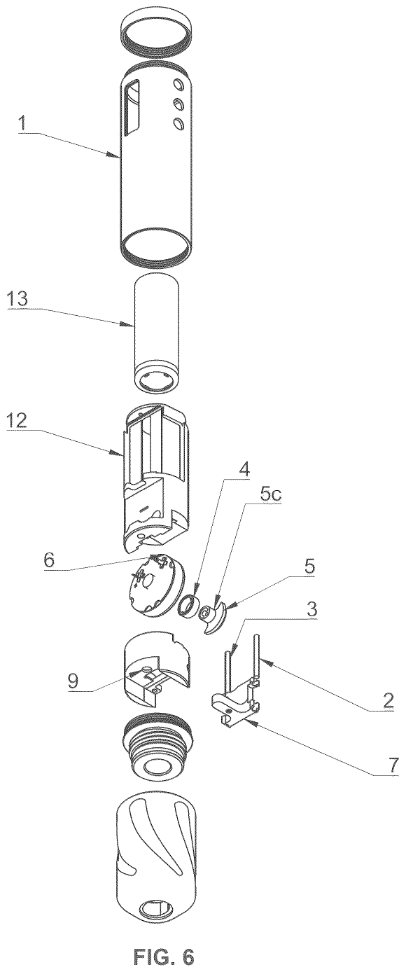

FIG. 6 is an exploded isometric view of the complete tattoo apparatus, showing the full assembly sequence and the spatial relationships among all principal components. The components are shown separated along the longitudinal axis of the apparatus in the order in which they are assembled.

At the uppermost position in FIG. 6, an end cap is shown above the housing 1. The end cap closes the upper end of the housing 1 and may be removable to permit access to the battery cell 13 for replacement.

The housing 1 is the main structural body of the apparatus, formed as an elongated tubular enclosure. The housing 1 includes a series of apertures formed in its wall, which may accommodate a power switch, an indicator light, ventilation, or a combination thereof. The housing 1 defines the external form factor of the apparatus and provides the primary grip surface for the operator during use.

The battery cell 13 is shown below the housing 1 in the exploded sequence, confirming the insertion arrangement described with reference to FIG. 5. The battery module frame 12 is shown below the battery cell 13, with its open-frame construction and rectangular window visible. In the assembled condition, the battery module frame 12 is received within the housing 1 and the battery cell 13 is inserted into the battery module frame 12.

The motor front plate 4 is shown below the battery module frame 12, confirming its position at the interface between the battery and power module region of the apparatus and the drive mechanism region. The motor 6 is shown below the motor front plate 4 as a generally cylindrical component with its output shaft extending downwardly toward the eccentric body 5. The eccentric body 5 is shown adjacent to the motor output shaft, with the offset bearing seat 5c visible as the eccentric boss portion that is spaced radially from the central bore.

The guide rods 2, 3 are shown extending downwardly from the motor and housing region, with the sliding block 7 mounted thereon. The sliding block 7 is shown in the exploded view with its rod-receiving bores and upper surface geometry visible, confirming the structural arrangement described with reference to FIGS. 1 through 2B.

A lower housing section 9 is shown below the sliding block 7. The lower housing section 9 forms the forward grip region of the apparatus and encloses the sliding block 7 and the lower portions of the guide rods 2, 3 in the assembled condition. The lower housing section 9 includes the magnet cavity described with reference to FIGS. 3 and 4A, which is formed in an inner wall of the lower housing section 9 in a position corresponding to the magnet cavity 8 of the sliding block 7 when the apparatus is fully assembled. In the assembled condition, the permanent magnets within the respective cavities 8 and 9 are brought into the facing like-pole arrangement described with reference to FIG. 3, generating the stabilising repulsive preload on the sliding block 7.

At the lowermost position in FIG. 6, a nose cone is shown below the lower housing section 9. The nose cone provides a tapered forward profile for the apparatus and may be removable to permit attachment of a tattoo needle cartridge to the distal end of the sliding block 7. In the assembled condition, the components shown in FIG. 6 are received within and secured to one another in the order depicted, forming a unitary handheld wireless tattoo apparatus having the drive mechanism at its operational core and the replaceable battery cell 13 providing electrical power to the motor 6.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A tattooing apparatus comprising:
a housing;
a motor disposed within the housing and having an output shaft;
a pair of guide rods fixed within the housing and arranged parallel to one another;
a sliding block mounted on the guide rods for linear reciprocation therealong, the sliding block having rod-receiving bores through which the guide rods pass;
an eccentric body mounted on the output shaft of the motor for co-rotation therewith, the eccentric body having a bearing seat that is radially offset from an axis of the output shaft;
a bearing mounted on the bearing seat of the eccentric body, the bearing having an outer ring that engages the sliding block such that rotation of the output shaft causes the bearing to orbit about the axis of the output shaft and thereby drive the sliding block in linear reciprocation along the guide rods;
a first permanent magnet disposed in the sliding block and a second permanent magnet disposed in the housing, the first and second permanent magnets being arranged in a like-pole repulsive orientation to generate a magnetic preload that stabilises the sliding block during reciprocation along the guide rods; and
a recess formed in an upper surface of the sliding block and configured to receive a portion of the bearing at a top-dead-centre position of the eccentric body, the recess having a profiled internal surface such that, during rotation of the eccentric body past the top-dead-centre position, engagement of the bearing with the profiled internal surface produces an increased downward acceleration of the sliding block relative to a sliding block lacking the recess.

2. The apparatus of claim 1, wherein the eccentric body has a central smooth bore configured to receive the output shaft, a lateral threaded hole oriented transversely to the central bore, and the bearing seat is formed as an offset cylindrical boss, and wherein a set screw is received in the lateral threaded hole and tightened against the output shaft to secure the eccentric body to the output shaft.

3. The apparatus of claim 1, wherein the sliding block is formed of a polymer composite material.

4. The apparatus of claim 3, wherein the polymer composite material comprises a polyoxymethylene and polytetrafluoroethylene composite.

5. The apparatus of claim 1, wherein the guide rods are cylindrical and formed of hardened stainless steel having a polished finish.

6. The apparatus of claim 1, wherein the rod-receiving bores of the sliding block define an operating clearance of between 0.03 mm and 0.08 mm relative to the guide rods.

7. The apparatus of claim 1, wherein the first permanent magnet is disposed within a first magnet cavity formed in the sliding block and the second permanent magnet is disposed within a second magnet cavity formed in an inner wall of the housing, the first and second magnet cavities being oriented to face one another when the sliding block is assembled within the housing.

8. The apparatus of claim 1, wherein the profiled internal surface of the recess comprises a profile selected from a circular arc, a spline-defined contour, a compound curve, an angled surface, and a stepped surface.

9. The apparatus of claim 1, wherein the recess is configured such that, at the top-dead-centre position, a contact force between the bearing and the sliding block is temporarily reduced to produce a transient floating condition in the sliding block prior to the increased downward acceleration.

10. The apparatus of claim 1, wherein the radial offset of the bearing seat from the axis of the output shaft defines a stroke length of between 2.5 mm and 5.5 mm.

11. The apparatus of claim 1, wherein the sliding block further includes a cartridge-engaging member at a distal end thereof, the cartridge-engaging member being configured to transfer linear reciprocation of the sliding block to a tattoo needle cartridge.

12. The apparatus of claim 1, further comprising a battery cell disposed within the housing and electrically connected to the motor, such that the apparatus is operable as a wireless handheld device.

13. The apparatus of claim 12, further comprising a battery module frame fixedly disposed within the housing and defining an internal receptacle configured to receive and locate the battery cell, wherein the battery cell is removable from the battery module frame for replacement by a user without disassembly of the drive mechanism.

14. A linear drive mechanism for converting rotary motion into linear reciprocation, the mechanism comprising:
a pair of guide rods arranged parallel to one another;
a sliding block mounted on the guide rods for linear reciprocation therealong;
an eccentric body configured to be mounted on a rotary output shaft, the eccentric body having a bearing seat that is radially offset from a rotational axis of the output shaft;
a bearing mounted on the bearing seat of the eccentric body, the bearing having an outer ring that engages the sliding block to convert rotation of the output shaft into linear reciprocation of the sliding block along the guide rods;
a magnetic preload system comprising a first permanent magnet associated with the sliding block and a second permanent magnet associated with a stationary structure adjacent to the sliding block, the first and second permanent magnets being arranged in a like-pole repulsive orientation to generate a stabilising force on the sliding block during reciprocation; and
a recess formed in a surface of the sliding block that faces the bearing, the recess being configured to receive a portion of the bearing at a top-dead-centre position of the eccentric body, the recess having a profiled internal surface that produces an increased acceleration of the sliding block away from the bearing during rotation of the eccentric body past the top-dead-centre position.

15. The mechanism of claim 14, wherein the eccentric body has a central smooth bore configured to receive the rotary output shaft, a lateral threaded hole for a set screw, and the bearing seat is formed as an offset cylindrical boss, and wherein the central bore, the lateral threaded hole, and the offset bearing seat are formed in a unitary body.

16. The mechanism of claim 14, wherein the sliding block is formed of a self-lubricating polymer composite and the guide rods are formed of hardened steel, such that the sliding block reciprocates on the guide rods without application of liquid lubricant.

17. The mechanism of claim 14, wherein the profiled internal surface of the recess is shaped such that the bearing momentarily reduces its contact force on the sliding block at the top-dead-centre position and subsequently applies an accelerating force to the sliding block during an initial portion of a downstroke.

18. A method of operating a tattoo apparatus, the method comprising:
rotating an output shaft of a motor disposed within a housing to rotate an eccentric body mounted on the output shaft, the eccentric body carrying a bearing on a bearing seat that is radially offset from an axis of the output shaft;
driving a sliding block in linear reciprocation along a pair of parallel guide rods fixed within the housing by engagement of an outer ring of the bearing with the sliding block as the bearing orbits about the axis of the output shaft;
stabilising the sliding block during reciprocation by a repulsive magnetic interaction between a first permanent magnet disposed in the sliding block and a second permanent magnet disposed in the housing, the first and second permanent magnets being arranged in a like-pole orientation; and
modifying an acceleration profile of the sliding block during a downstroke by receiving a portion of the bearing into a recess formed in an upper surface of the sliding block at a top-dead-centre position of the eccentric body, the recess having a profiled internal surface that produces an increased downward acceleration of the sliding block as the bearing transitions out of the recess during rotation past the top-dead-centre position.

19. The method of claim 18, wherein the step of modifying the acceleration profile includes producing a transient floating condition in the sliding block at the top-dead-centre position in which a contact force between the bearing and the sliding block is temporarily reduced, followed by the increased downward acceleration during an initial portion of the downstroke.

20. The method of claim 18, further comprising powering the motor from a replaceable battery cell disposed within the housing such that the apparatus operates as a cordless handheld device.
